(19) United States
(12) Patent Application Publication
(10) Pub. No.: US 2025/0032169 A1
(43) Pub. Date: Jan. 30, 2025

(54) Treatment tool

(71) Applicant: OLYMPUS MEDICAL SYSTEMS CORP., Tokyo (JP)

(72) Inventor: Tsunetaka Akagane, Tokyo (JP)

(21) Appl. No.: 18/778,271

(22) Filed: Jul. 19, 2024

Related U.S. Application Data
(60) Provisional application No. 63/515,461, filed on Jul. 25, 2023.

(51) Int. Cl. A61B 18/14; A61B 17/32; A61B 18/00

(52) U.S. Cl. CPC A61B 18/1445; A61B 17/320092; A61B 2017/320095; A61B 2018/00077; A61B 2018/00083; A61B 2018/00107; A61B 2018/00589; A61B 2018/0063; A61B 2018/1467

(57) ABSTRACT

A treatment tool can include a first jaw including a first electrode, and a second jaw configured to be opened and closed with respect to the first jaw, and the second jaw including a second electrode. The first electrode and the second electrode are configured to cause a high-frequency current, and at least a part of the first jaw includes a first portion having an uneven surface.

### CROSS-REFERENCE TO RELATED APPLICATION

This application is based on and claims priority under 35 U.S.C. § 119 to U.S. Provisional Application No. 63/515,461, filed Jul. 25, 2023, the entire contents of which are incorporated herein by reference.

### TECHNICAL FIELD

The present disclosure relates to a treatment tool.

### BACKGROUND

A treatment tool can apply high-frequency energy to a site to be treated (a treatment target) in a living tissue and can treat the treatment target (such as by delivering energy) An example of such a treatment tool is described in Japanese Laid-open Patent Publication No. 2008-11987. Such a treatment tool can uniformly apply the high-frequency energy to the treatment target.

### SUMMARY

In some embodiments, a treatment tool can include a first jaw including a first electrode, and a second jaw configured to be opened and closed with respect to the first jaw, and the second jaw including a second electrode. The first electrode and the second electrode are configured to cause a high-frequency current, and at least a part of the first jaw includes a first portion having an uneven surface.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a diagram illustrating a treatment system according to an embodiment;

FIG. 2 is a diagram for explaining a configuration of a distal end portion of the treatment tool;

FIG. 3 is a diagram for explaining configurations of a jaw and a vibration transmitter;

FIG. 4 is a diagram for explaining a configuration of seal strengthening portions;

FIG. 5 is a diagram illustrating a first modification of an embodiment;

FIG. 6 is a diagram illustrating a second modification of an embodiment;

FIG. 7 is a diagram illustrating a third modification of an embodiment;

FIG. 8 is a diagram illustrating a fourth modification of an embodiment;

FIG. 9 is a diagram illustrating a fifth modification of an embodiment;

FIG. 10 is a diagram illustrating the fifth modification of an embodiment; and

FIG. 11 is a diagram illustrating the fifth modification of an embodiment.

### DETAILED DESCRIPTION

Modes (hereinafter, embodiments) for carrying out the disclosure will be described below with reference to the drawings. Meanwhile, the disclosure is not limited by the embodiments described below. In addition, in description of the drawings, the same or similar components are denoted by the same reference symbols.

### Overall Configuration of Treatment System

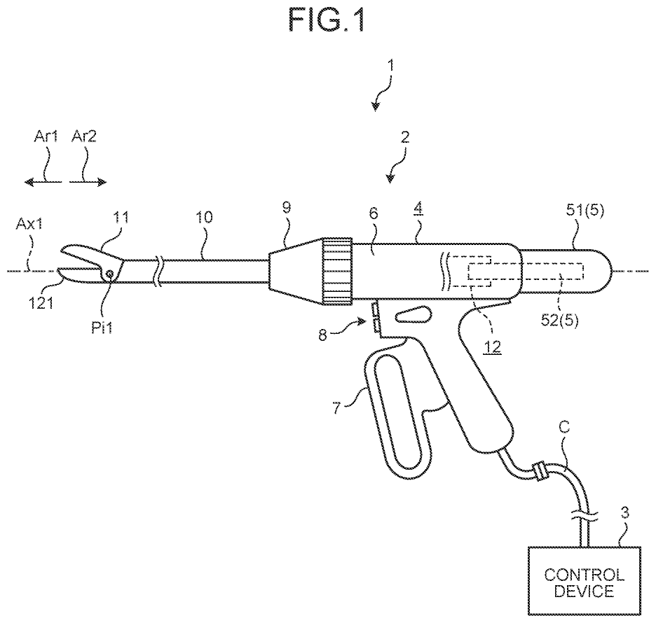

FIG. 1 is a diagram illustrating a treatment system 1 according to an embodiment. The treatment system 1 can apply treatment energy to a site to be treated (hereinafter, described as a treatment target) in a living tissue and can treat the treatment target. The treatment energy can include at least one of ultrasonic energy or high-frequency energy. Further, a process that can be performed by the treatment system 1 according to an embodiment is certain treatment, such as coagulation (sealing) of the treatment target or incision of the treatment target. Furthermore, it may be possible to perform coagulation and incision at the same time. Meanwhile, the treatment energy that is applied to the treatment target need not always be both of the ultrasonic energy and the high-frequency energy but can be only the ultrasonic energy or only the high-frequency energy. The treatment system 1 can include, as illustrated in FIG. 1, a treatment tool 2 and a control device 3.

### Configuration of Treatment Tool

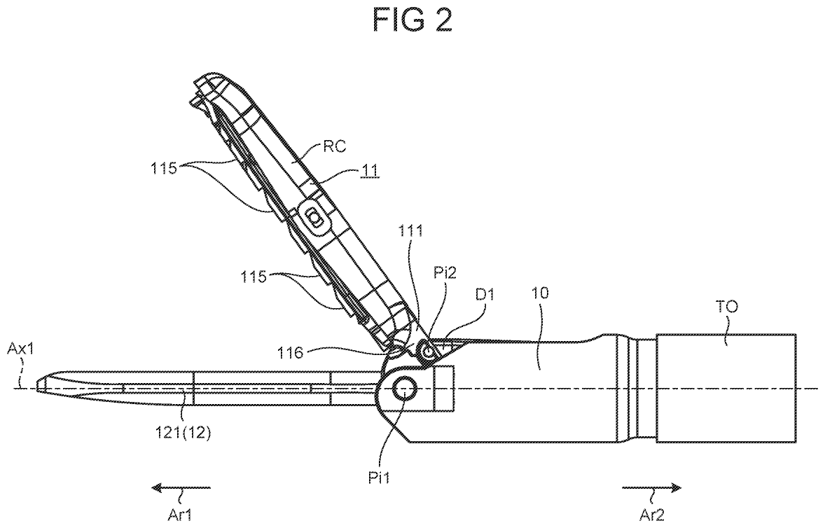

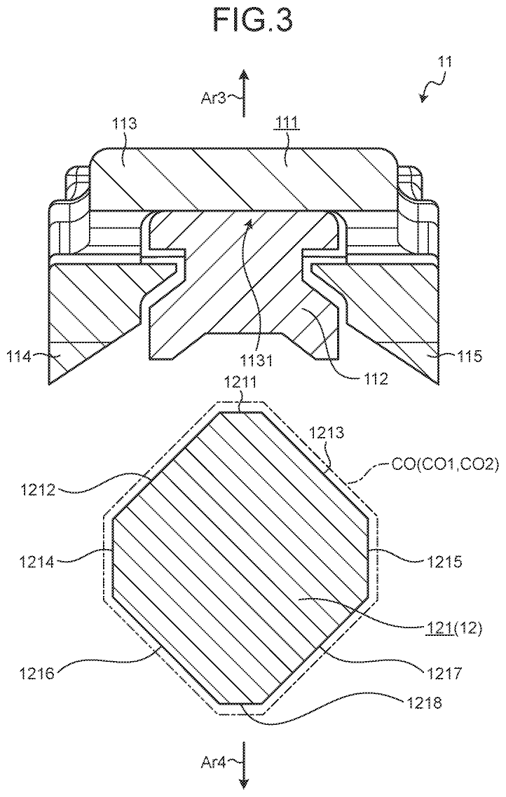

In the following, one side along a central axis Aₓ₁ of a sheath 10 is described as a distal end side Aᵣ₁, and the other side is described as a proximal end side Aᵣ₂ (FIG. 1). Further, a “width direction” described below indicates a direction that is perpendicular to an open-close direction of a jaw 11 (second jaw) with respect to the central axis Aₓ₁ and a treatment portion 121 (first jaw), that is perpendicular to the sheets of FIG. 1 and FIG. 2, and that is a horizontal direction in FIG. 3. FIG. 2 is a diagram for explaining a configuration of a distal end portion of the treatment tool 2. Specifically, FIG. 2 illustrates a view of the distal end portion of the treatment tool 2 viewed along the width direction. The treatment tool 2 is a treatment tool that can apply at least one of ultrasonic energy or high-frequency energy to a treatment target and can treat the treatment target. The treatment tool 2 can include, as illustrated in FIG. 1, a hand piece 4 and an ultrasonic transducer 5.

The treatment tool 2 comprises the first jaw 121 including the first electrode 121, and the second jaw 11 configured to be opened and closed with respect to the first jaw 121, and the second jaw 11 including the second electrode 111. The first electrode 121 and the second electrode 111 are configured to cause a high-frequency current, and at least a part of the first jaw 121 includes the first portion 122 having an uneven surface. The first jaw can be configured to move with respect to the second jaw. The first jaw can be configured to pivotably rotate with respect to the second jaw.

The hand piece 4 can include, as illustrated in FIG. 1 to FIG. 3, a holder case 6 (FIG. 1), an operation handle 7 (handle) (FIG. 1), switches 8 (FIG. 1), a rotary knob 9 (FIG. 1), the sheath 10, the jaw 11, and a vibration transmitter 12.

The holder case 6 can support the entire treatment tool 2.

The operation handle 7 can be attached to the holder case 6 in a movable manner

and can receive an open-close operation performed by an operating person (e.g., a user such as a physician or another operator). The switches 8 can be arranged so as to be exposed to outside of the holder case 6 and receive treatment operation that is performed by the operating person. The rotary knob 9 can be an approximately cylindrical shape that is coaxial with the central axis Aₓ₁ and can be arranged on the distal end side Aᵣ₁ of the holder case 6. The handle 7 is configured to open and close the second jaw 11 with respect to the first jaw 121. The approximately cylindrical shape includes a shape that can achieve approximately the same effect, and design error margins. Further, the rotary knob 9 can receive a rotation operation that is performed by the operating person. Due to the rotation operation, the rotary knob 9 can rotate about the central axis Aₓ₁ with respect to the holder case 6. Furthermore, with the rotation of the rotary knob 9, the sheath 10, the jaw 11, and the vibration transmitter 12 can rotate about the central axis Aₓ₁.

The sheath 10 can include a cylindrical pipe that is configured with an electrically conductive material, such as a metal. A first pin Pᵢ₁ that has a cylindrical shape and that extends in the width direction (FIG. 1 and FIG. 2) can be fixed to an end portion of the sheath 10 on the distal end side Aᵣ₁. Further, an outer peripheral surface of the sheath 10 can be covered by an outer tube Tₒ (FIG. 2) that has electrical insulation property. Furthermore, an inner peripheral surface of the sheath 10 can be covered by an inner tube that has electrical insulation property.

FIG. 3 is a diagram for explaining configurations of the jaw 11 and the vibration transmitter 12. Specifically, FIG. 3 is a cross-sectional view of the jaw 11 and the vibration transmitter 12 cut along a plane that is perpendicular to the central axis Aₓ₁. Meanwhile, in FIG. 3, illustration of a cover RC is omitted for convenience of explanation. In the following, for explanation of the configuration of the jaw 11, a side that is located away from the treatment portion 121 will be described as a back side Aᵣ₃, and a side that is located close to the treatment portion 121 will be described as a treatment portion side Aᵣ₄.

The jaw 11 corresponds to a second jaw. The jaw 11 can be pivotally supported by the first pin Pᵢ₁ with respect to the end portion of the sheath 10 on the distal end side Aᵣ₁ and can be configured so as to be rotatable about a central axis of the first pin Pᵢ₁ (an axis along a direction perpendicular to the sheets of FIG. 1 and FIG. 2). Further, by rotating about the central axis of the first pin Pᵢ₁, the jaw 11 can be opened and closed with respect to the treatment portion 121 that is arranged on an end portion of the vibration transmitter 12 on the distal end side Aᵣ₁. When the jaw 11 is closed with respect to the treatment portion 121, the treatment target is gripped between the jaw 11 and the treatment portion 121.

The first jaw 121 can comprise a first surface (1211, 1212, 1213) and a second surface (1216, 1217, 1218). The first surface (1211, 1212, 1213) can be configured to face the second jaw 11, and the first surface (1211, 1212, 1213) has the first portion 122, and the second surface (1216, 1217, 1218) can be configured not to face the second jaw 11, and the second surface (1216, 1217, 1218) can have electrically insulating material. The first jaw can comprise a third surface (1214, 1215) connecting the first surface (1211, 1212, 1213) and the second surface (1216, 1217, 1218), the third surface (1214, 1215) does not contact with the second jaw 11 when the second jaw 11 is closed with respect to the first jaw 121, and wherein the first surface (1211, 1212, 1213) contacts with the second jaw 11 when the second jaw 11 is closed with respect to the first jaw 121.

The first surface (1211, 1212, 1213) can comprise a top surface 1121, a first and second inclined surface 1212, 1213, the first and second inclined surfaces 1212, 1213 are inclined with respect to the top surface 1121. Both of the first and second inclined surfaces can have the first portion. Furthermore, the top surface 1211 can have the first portion. Alternatively, only the top surface 1211 can have the first portion, and the first and second inclined surfaces don't have the first portion.

The jaw 11 can include, as illustrated in FIG. 3, a jaw main body 111 and a pad 112. The jaw main body 111 can be configured with an electrically conductive material. The jaw main body 111 can be a member in which, as illustrated in FIG. 2 and FIG. 3, can include a base 113 (FIG. 3), a plurality of first teeth portions 114 (FIG. 3), a plurality of second teeth portions 115 (FIG. 3), and a bearing portion 116 (FIG. 2).

The base 113 can be configured with an elongated plate-like body. In an embodiment, a longitudinal direction of the base 113 is a direction that extends along a curve that is oriented to the left while approaching to the distal end side Aᵣ₁ when viewed from the proximal end side Aᵣ₂ in a state in which the jaw 11 is located on an upper side of the treatment portion 121.

On a surface of the base 113 on the back side Aᵣ₃, the cover RC (FIG. 2) can be configured with resin that has electrical insulation property can be integrally formed so as to cover the surface on the back side Aᵣ₃. Meanwhile, in an embodiment, the cover RC can be formed by insert-molding with respect to the base 113, but embodiments are not limited to this example. For example, it may be possible to adopt a configuration in which the cover RC is fixed to the base 113 by snap fit or a metal pin.

The plurality of first teeth portions 114 can protrude from one end side of a surface of the base 113 in the width direction on the treatment portion side Aᵣ₄ toward the treatment portion side Aᵣ₄ and can be arranged parallel to one another along the longitudinal direction of the base 113.

The plurality of second teeth portions 115 can protrude from the other end side of the surface of the base 113 in the width direction on the treatment portion side Aᵣ₄ toward the treatment portion side Aᵣ₄ and are arranged parallel to one another along the longitudinal direction of the base 113.

Here, on the surface of the base 113 on the treatment portion side Aᵣ₄, in a center portion in the width direction between the plurality of first teeth portions 114 and the plurality of second teeth portions 115, as illustrated in FIG. 3, a recessed portion 1131 that is recessed toward the back side Aᵣ₃ and that extends along the longitudinal direction of the base 113 can be arranged.

The bearing portion 116 can be a portion that is arranged on a proximal end of the base 113 and pivotally supported by the first pin Pᵢ₁ with respect to the sheath 10. Furthermore, a cylindrical second pin Pᵢ₂ (FIG. 2) that extends in the width direction can be fixed to the bearing portion 116 by welding. The second pin Pᵢ₂ can be connected to an open-close mechanism D1 (FIG. 2) that can be inserted in the sheath 10. Moreover, the jaw 11 can rotate about the central axis of the first pin Pᵢ₁ in conjunction with movement of the open-close mechanism D1 to the distal end side Aᵣ₁ or the proximal end side Aᵣ₂ in accordance with open-close operation that is performed by an operating person, such as a physician or other operator, and can be opened or closed with respect to the treatment portion 121.

The pad 112 can be configured or coated with a resin material, such as polytetrafluoroethylene (PTFE), that has electrical insulation property and biocompatibility. The pad 112 can have an approximately rectangular shape that extends along the longitudinal direction of the base 113. The approximately rectangular includes a shape that can achieve approximately the same effect, and design error margins. Furthermore, the pad 112 can be fixed to the recessed portion 1131 in the base 113 as illustrated in FIG. 3. Moreover, the pad 112 can come into contact with or make contact with the treatment portion 121 when the jaw 11 is brought close to the treatment portion 121.

The vibration transmitter 12 can have or be formed in an elongated shape and can configured with or formed with an electrically conductive material. Further, as illustrated in FIG. 2, the vibration transmitter 12 can be inserted in the sheath 10 such that the treatment portion 121 protrudes to the outside. In this case, an end portion of the vibration transmitter 12 on the proximal end side Aᵣ₂ can be mechanically connected to an ultrasonic transducer portion 52 that constitutes the ultrasonic transducer 5 as illustrated in FIG. 1. Furthermore, the vibration transmitter 12 can transmit ultrasonic vibration that is generated by the ultrasonic transducer 5 from the end portion on the proximal end side Aᵣ₂ to the treatment portion 121. In an embodiment, the ultrasonic vibration can be longitudinal vibration that vibrates in a direction extending along the central axis Aₓ₁. The vibration transmitter 12 is configured to treat the treatment target by ultrasonic vibration.

In an embodiment, similarly to the jaw 11, the treatment portion 121 can extend along a curve that is oriented to the left while approaching to the distal end side Aᵣ₁ when viewed from the proximal end side Aᵣ₂ in a state in which the jaw 11 is located on the upper side of the treatment portion 121. Furthermore, as illustrated in FIG. 3, a cross-section of the treatment portion 121 cut along the plane that is perpendicular to the central axis Aₓ₁ can have an approximately octagonal shape. The approximately octagonal includes a shape that can achieve approximately the same effect, and design error margins. Meanwhile, the octagonal shape as the cross-sectional shape of the treatment portion 121 is one example, and it may be possible to adopt a different shape, such as a circular shape. In the following, for convenience of explanation, explanation will be given based on the assumption that the cross-sectional shape of the treatment portion 121 is the octagonal shape.

In the following, a flat surface of the treatment portion 121 located at the side of the jaw 11 will be described as a first surface 1211. The first surface 1211 is a surface that can come into contact with the pad 112 when the jaw 11 is closed with respect to the treatment portion 121. In other words, the first surface 1211 can correspond to a first contact portion. Furthermore, surfaces that are adjacent to the first surface 1211 in a circumferential direction about the central axis of the treatment portion 121 will be described as a second surface 1212 and a third surface 1213. Moreover, the second surface 1212 and the third surface 1213 can correspond to first non-contact portions. Furthermore, surfaces that are adjacent to the second surface 1212 and the third surface 1213 in the circumferential direction about the central axis of the treatment portion 121 will be described as a fourth surface 1214 and a fifth surface 1215. Moreover, surfaces that are connected to the fourth surface 1214 and the fifth surface 1215 in the circumferential direction about the central axis of the treatment portion 121 will be described as a sixth surface 1216 and a seventh surface 1217. Furthermore, a surface that is located between the sixth surface 1216 and the seventh surface 1217 and serves as a back side of the first surface 1211 will be described as an eighth surface 1218.

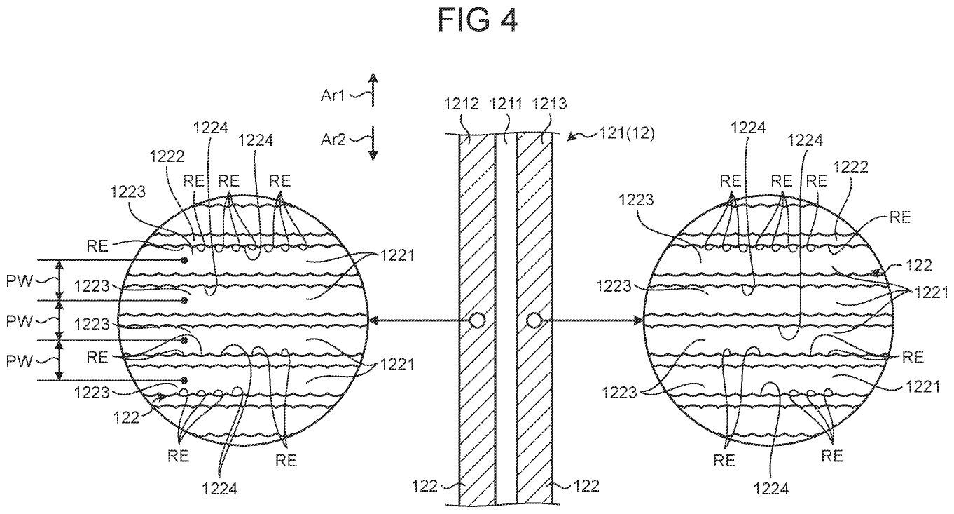

Moreover, in an embodiment, first seal strengthening portions 122 (first portion) (see FIG. 4) can be arranged on the second surface 1212 and the third surface 1213 among the surfaces of the treatment portion 121. Meanwhile, a specific configuration of the first seal strengthening portions 122 will be described in the section of “Configuration of first seal strengthening portions” to be described later.

Furthermore, in an embodiment, a coating layer CO (FIG. 3) can be arranged on the surfaces of the treatment portion 121. A specific configuration of the coating layer CO will be described in the section of “Configuration of coating layer” to be described later.

The ultrasonic transducer 5 can include, as illustrated in FIG. 1, a transducer (TD) case 51 and the ultrasonic transducer portion 52. The TD case 51 can support the ultrasonic transducer portion 52 and can be detachably connected to the holder case 6. The ultrasonic transducer portion 52 can generate ultrasonic vibration under the control of the control device 3. In an example, the ultrasonic transducer portion 52 can be configured with a bolt-clamped Langevin-type transducer (BLT).

### Configuration of Control Device

The control device 3 can comprehensively control operation of the treatment tool 2 via an electrical cable C (FIG. 1). Specifically, the control device 3 can detect, via the electrical cable C, a treatment operation that is performed by the operating person on the switches 8. Furthermore, when detecting the treatment operation, the control device 3 can apply, via the electrical cable C, treatment energy to a treatment target gripped between the jaw 11 and the treatment portion 121. In other words, the control device 3 can treat the treatment target. For example, when applying the ultrasonic energy to the treatment target, the control device 3 can supply, via the electrical cable C, driving power to the ultrasonic transducer portion 52. Accordingly, the ultrasonic transducer portion 52 can generate longitudinal vibration (ultrasonic vibration) that is vibration in a direction along the central axis Aₓ₁. Further, the treatment portion 121 can vibrate at a predetermined amplitude due to the longitudinal vibration. The ultrasonic vibration can be applied from the treatment portion 121 to the treatment target that is gripped between the jaw 11 (the pad 112) and the treatment portion 121. In other words, ultrasonic energy can be applied from the treatment portion 121 to the treatment target.

Moreover, for example, when applying high-frequency energy to the treatment target, the control device 3 can supply, via the electrical cable C, high-frequency power between the jaw main body 111 and the vibration transmitter 12. In an example, when the high-frequency power is supplied between the jaw main body 111 and the vibration transmitter 12, a high-frequency current can flow to the treatment target that is located between the jaw main body 111 and the treatment portion 121. Hence, the high-frequency energy can be applied to the treatment target.

As described above, in an embodiment, the treatment portion 121 can function or operate as a first electrode. Further, the jaw main body 111 can function or operate as a second electrode.

### Configuration of First Seal Strengthening Portions

A configuration of the first seal strengthening portions 122 will be described below. FIG. 4 is a diagram for explaining the configuration of the first seal strengthening portions 122. Specifically, FIG. 4 is a diagram of the vibration transmitter 12 that is viewed at the side of the first surface 1211 along a normal direction of the first surface 1211. Meanwhile, in FIG. 4, portions in which the first seal strengthening portions 122 are arranged are indicated by diagonal lines for convenience of explanation. The first seal strengthening portions 122 are portions that can be formed in uneven shapes. In an example, the first seal strengthening portions 122 can be formed by arranging a plurality of circular recessed portions RE in the second surface 1212 and the third surface 1213 by laser processing using a short pulse laser as illustrated in FIG. 4. In the example illustrated in FIG. 4, a plurality of rows of the recessed portions RE (hereinafter, described as grooves 1221), in each of which the plurality of recessed portions RE are arranged in an overlapping manner (in a communicating manner) in a direction perpendicular to the longitudinal direction of the vibration transmitter 12, can be arranged parallel to each other along the longitudinal direction.

Furthermore, in an embodiment, each of depth dimensions and width dimensions of the grooves 1221 and pitch dimensions (pitch widths PW (FIG. 4)) between the plurality of grooves 1221 can be set to be equal to or smaller than 100 micrometers (μm). Meanwhile, the depth dimensions, the width dimensions, and the pitch widths PW of the grooves 1221 can be set to the same value or different values. A thickness of the first portion 122 is equal to or smaller than 100 micrometers (μm). The first portion 122 can include a plurality of grooves 1221 each extending in a direction intersecting the longitudinal direction of the first jaw 121. The direction can be orthogonal to the longitudinal direction. The direction is not parallel to the longitudinal axis of the first jaw. Each groove of the plurality of grooves can have side surfaces having a plurality of recesses RE recessed in a direction intersecting with the longitudinal direction. The side surfaces of the groove can have an uneven surface. Each uneven surface can oppose each other. The plurality of grooves can comprise a top surface 1222, a bottom surface, and a side surface connecting the top surface and the bottom surface. An area of the top surface of the groove can be smaller than an area of the bottom surface of the groove. A distance between each of the top surfaces can be set to be equal to or smaller than 100 micrometers (μm). A distance between each of the side surfaces can be set to be equal to or smaller than 100 micrometers (μm).

Meanwhile, in the following, portions other than the portions in which the grooves 1221 are formed on the second surface 1212 and the third surface 1213 will be described as top surfaces 1222 (FIG. 4), bottom surfaces of the grooves 1221 will be described as bottom surfaces 1223 (FIG. 4), and side surfaces of the grooves 1221 will be described as side surfaces 1224 (FIG. 4). Furthermore, in an embodiment, as described above, the first seal strengthening portions 122 can be configured by laser processing, and therefore, each of the top surfaces 1222, the bottom surfaces 1223, and the side surfaces 1224 can be formed in an uneven shape.

### Configuration of Coating Layer

A configuration of the coating layer CO will be described below. The coating layer CO includes, as illustrated in FIG. 3, a first coating layer CO₁ and a second coating layer CO₂. The first coating layer CO₁ is a coating layer that can be configured with a water-repellent material, such as a fluorine coating agent or a siloxane coating agent. In an example, the first coating layer CO₁ can be arranged so as to cover the first surface 1211 to the third surface 1213 among the surfaces of the treatment portion 121. At least a part of the first surface can have a water-repellent material.

In an example, a thickness dimension of the first coating layer CO₁ that is arranged on the surfaces on which the first seal strengthening portions 122 are arranged can be set equal to or smaller than 10% of the depth dimension of each of the first seal strengthening portions 122. In an example, the thickness dimension can be set equal to or smaller than 1 μm.

The second coating layer CO₂ is a coating layer that can be configured with or coated with an electrically insulating material, such as polyetheretherketone (PEEK) that is resin including both of an ether group and a ketone group. The second coating layer CO₂ can be arranged so as to cover the fourth surface 1214 to the eighth surface 1218 among the surfaces of the treatment portion 121.

In an example, the first seal strengthening portions 122 as described above can be arranged on the surfaces of the treatment portion 121. The first seal strengthening portions 122 that have uneven shapes can be arranged on the surfaces of the treatment portion 121. With the uneven shapes, surface areas of the first seal strengthening portions 122 per unit area are increased as compared to a surface area of a portion in which the uneven shape is not arranged. With this configuration, contact surfaces with the treatment target are increased by the first seal strengthening portions 122, so that it is possible to effectively apply high-frequency energy from the contact portions to the treatment target. Stated differently, it is possible to effectively apply high-frequency energy to an intended portion of the treatment target. Therefore, according to the treatment tool 2 of an embodiment, it is possible to improve sealing performance (treatment performance) on the treatment target, such as a blood vessel.

Meanwhile, the treatment target that is gripped between the jaw 11 and the treatment portion 121 can shrink in the longitudinal direction of the vibration transmitter 12 due to application of the treatment energy. When the treatment target shrinks as described above, it can be difficult to apply high-frequency energy to an intended portion of the treatment target. In the treatment tool 2 according to an embodiment, the first seal strengthening portions 122 can include the plurality of grooves 1221 that extend in a direction that crosses the longitudinal direction of the vibration transmitter 12. Therefore, the first seal strengthening portions 122 can function or operate as anti-slip members that restrict shrinkage of the treatment target due to application of the treatment energy as described above. Consequently, it is possible to apply high-frequency energy while the treatment target is fixed, so that it is possible to effectively apply the high-frequency energy to an intended potion of the treatment target and further improve the sealing performance on the treatment target.

In an example, the grooves 1221 are grooves in which the plurality of recessed portions RE that are formed by laser processing overlap with one another in a predetermined direction. The grooves 1221 comprise a regular pattern. Therefore, it is possible to form the top surfaces 1222, the bottom surfaces 1223, and the side surfaces 1224 in uneven shapes, so that it is possible to further increase the surface areas of the portions in which the first seal strengthening portions 122 are arranged and it is possible to effectively apply high-frequency energy from the portions to the treatment target.

### Other Embodiments

While the embodiments of the disclosure have been described above, the disclosure is not limited to the embodiments as described above. In an example, at least one of the first coating layer CO₁ and the second coating layer CO₂ need not be arranged on the treatment portion 121. In another example, it may be possible to arrange the first coating layer CO₁ on only the surfaces (the second surface 1212 and the third surface 1213) on which the first seal strengthening portions 122 are arranged. In another example, it may be possible to arrange the first coating layer CO₁, instead of the second coating layer CO₂, on the fourth surface 1214 and the fifth surface 1215. In another example, the open-close mechanism D1 can be arranged inside the sheath 10, but embodiments are not limited to this example. For example, it may be possible to adopt a configuration in which the sheath 10 itself moves to the distal end side Aᵣ₁ or the proximal end side Aᵣ₂ to open and close the jaw 11 with respect to the treatment portion 121. Furthermore, as for opening and closing of the jaw 11, the jaw 11 can be configured to be closed with respect to the treatment portion 121 when the open-close mechanism D1 or the sheath 10 moves to the distal end side Aᵣ₁ or can be configured to be closed with respect to the treatment portion 121 when the open-close mechanism D1 or the sheath 10 moves to the proximal end side Aᵣ₂.

In another, the ultrasonic transducer 5 can be configured so as to be detachable from the hand piece 4, but embodiments are not limited to this example, and it may be possible to adopt a configuration in which the ultrasonic transducer 5 is incorporated in the hand piece 4. In an example, the number of the switches 8 is not limited to two as illustrated in FIG. 1 but can be a single switch or can be three or more. In an example, when high-frequency power is supplied between the jaw 11 and the vibration transmitter 12, it may be possible to adopt reference voltage at the side of the jaw 11, or it may be possible to adopt reference voltage at the side of the vibration transmitter 12.

In an embodiment as described above, it may be possible to adopt a first modification to a fifth modification as described below.

First Modification

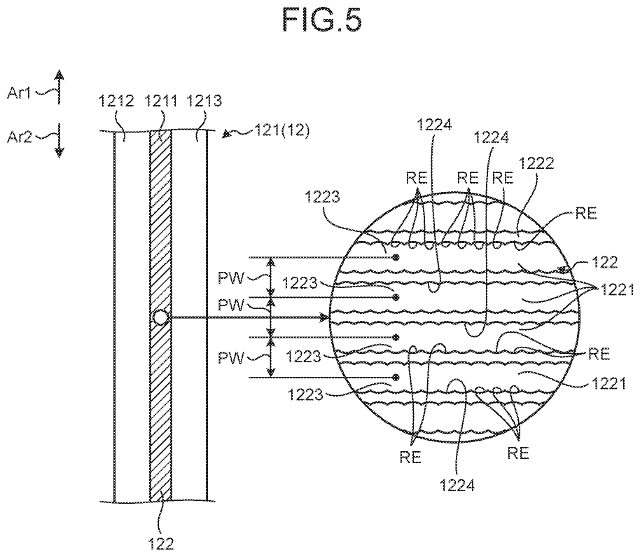

FIG. 5 is a diagram illustrating a first modification of an embodiment. Specifically, FIG. 5 is a diagram corresponding to FIG. 4. As illustrated in, in FIG. 5, a portion in which the first seal strengthening portion 122 is arranged is indicated by diagonal lines for convenience of explanation. In an example, the first seal strengthening portions 122 can be arranged on the second surface 1212 and the third surface 1213 among the surfaces of the treatment portion 121, but embodiments are not limited to this example, and it may be possible to arrange the first seal strengthening portion 122 on the first surface 1211 as in the first modification illustrated in FIG. 5. Even when the configuration of the first modification as described above is adopted, it can achieve effects described above.

### Second Modification

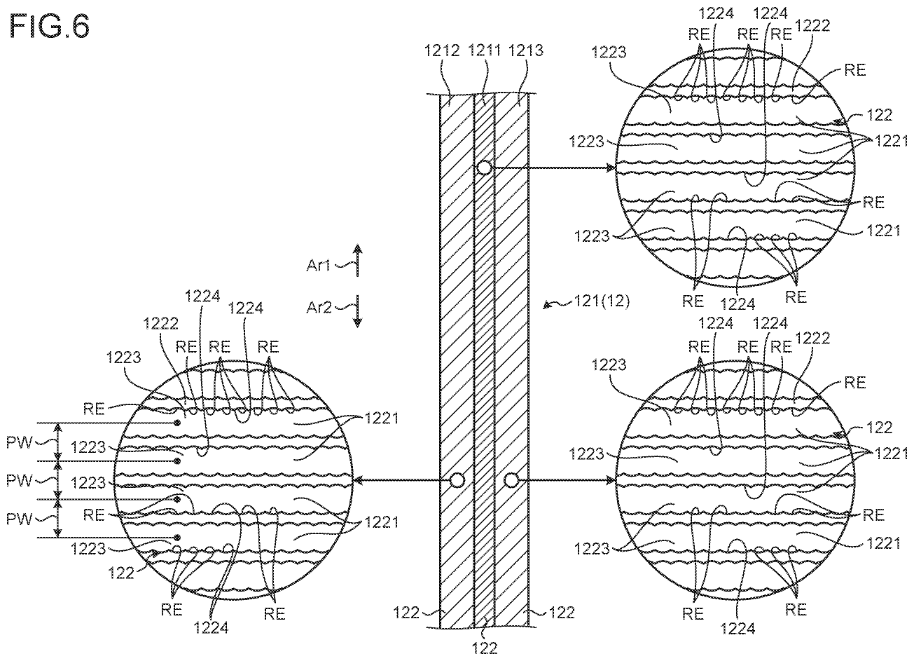

FIG. 6 is a diagram illustrating a second modification of an embodiment. Specifically, FIG. 6 is a diagram corresponding to FIG. 4. As illustrated in FIG. 6, portions in which the first seal strengthening portions 122 are arranged are indicated by diagonal lines for convenience of explanation.

In an example, the first seal strengthening portions 122 can be arranged on the second surface 1212 and the third surface 1213 among the surfaces of the treatment portion 121, but embodiments are not limited to this example, and it may be possible to arrange the first seal strengthening portions 122 on the first surface 1211 to the third surface 1213 as in the second modification illustrated in FIG. 6. Even when the configuration of the second modification as described above is adopted, it can achieve effects described above.

### Third Modification

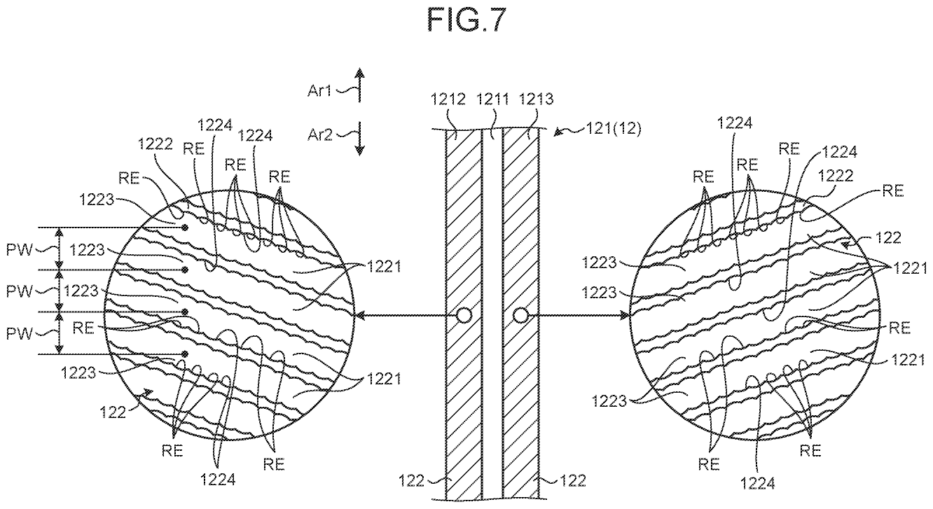

FIG. 7 is a diagram illustrating a third modification of an embodiment. Specifically, FIG. 7 is a diagram corresponding to FIG. 4. As illustrated in FIG. 7, portions in which the first seal strengthening portions 122 are arranged are indicated by diagonal lines for convenience of explanation.

In an example, the plurality of grooves 1221 that constitute the first seal strengthening portions 122 can be arranged so as to extend in the direction perpendicular to the longitudinal direction of the vibration transmitter 12 and so as to be parallel to one another along the longitudinal direction, but embodiments are not limited to this example.

The plurality of grooves 1221 can be arranged, for example, so as to extend along a direction at an angle other than 90° with respect to the longitudinal direction as in the third modification illustrated in FIG. 7 as long as the direction crosses the longitudinal direction of the vibration transmitter 12 and so as to be parallel to one another along the longitudinal direction. The first jaw 121 can include a first portion and a second portion. The first portion includes a plurality of first grooves each extending in a first direction intersecting the longitudinal direction of the first jaw 121. The second portion includes a plurality of second grooves each extending in a second direction intersecting the longitudinal direction of the first jaw 121. The first direction is symmetric with the second direction with respect to a longitudinal axis of the first jaw 121.

Even when the configuration of the third modification as described above is adopted, it can achieve effects described above.

### Fourth Modification

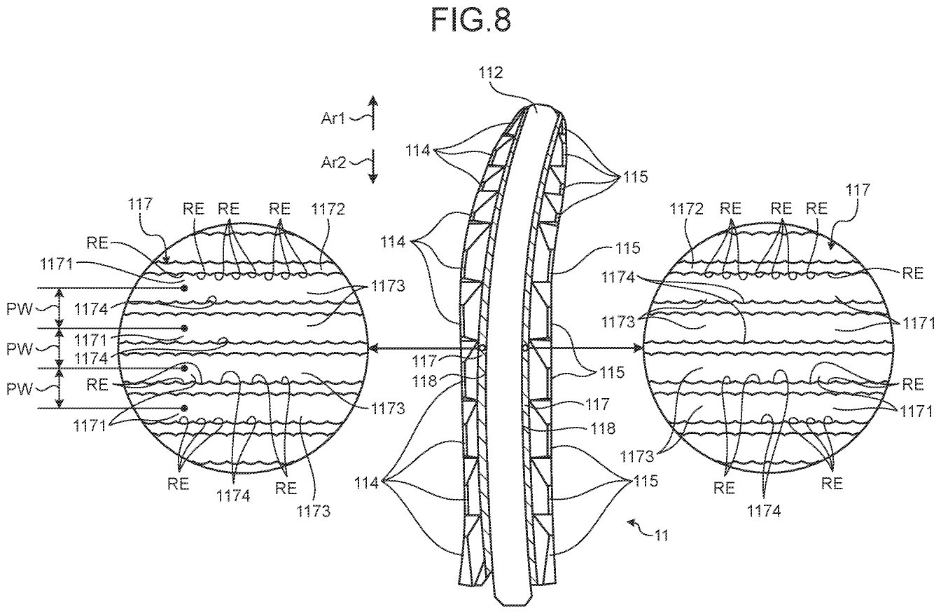

FIG. 8 is a diagram illustrating a fourth modification of an embodiment. Specifically, FIG. 8 illustrates the jaw 11 when viewed from the treatment portion side Ar4. As illustrated in FIG. 8, for convenience of explanation, portions in which second seal strengthening portions 117 are arranged are indicated by diagonal lines. In an example, as in the fourth modification illustrated in FIG. 8, it may be possible to arrange the second seal strengthening portions 117 (second portion) in the jaw 11. The uneven surface of the first jaw 121 can comprise a first uneven surface, and at least a part of the second jaw 11 can include a second portion 117 having a second uneven surface. The second electrode 111 can comprise a first surface 118 and a second surface 118. The first surface 118 is configured not to contact with the first jaw 121 when the second jaw 11 is closed with respect to the first jaw 121, and the second surface 118 is configured not to contact with the first jaw 121, the second surface 118 has the second portion 117 when the second jaw 11 is closed with respect to the first jaw 121.

The second seal strengthening portions 117 can be arranged in a pair of regions 118 located adjacent to the pad 112 and are arranged between the pad 112 and the plurality of first teeth portions 114 and the plurality of second teeth portions 115 on the treatment portion side Ar4 of the jaw 11 as illustrated in FIG. 8. Here, the pad 112 corresponds to a second contact portion. Furthermore, the pair of regions 118 corresponds to second non-contact portions.

The second seal strengthening portions 117 can be portions that are formed in uneven shapes, similarly to the first seal strengthening portions 122. In the fourth modification, the second seal strengthening portions 117 can be formed by arranging a plurality of circular recessed portions RE in the pair of regions 118 by laser processing using a short pulse laser as illustrated in FIG. 8. In the example illustrated in FIG. 8, a plurality of rows of the recessed portions RE (hereinafter, described as grooves 1171), in each of which the plurality of recessed portions RE are arranged in an overlapping manner (in a communicating manner) in a direction perpendicular to the longitudinal direction of the jaw 11, can be arranged parallel to one another along the longitudinal direction.

Furthermore, in the fourth modification, each of depth dimensions and width dimensions of the grooves 1171 and interval dimensions (pitch widths PW (FIG. 8)) between the plurality of grooves 1171 can be set to be equal to or smaller than 100 μm. Meanwhile, the depth dimensions, the width dimensions, and the pitch widths PW of the grooves 1171 can be set to the same value or different values.

In the pair of regions 118, portions other than the portions in which the grooves 1171 are formed will be described as top surfaces 1172 (FIG. 8), bottom surfaces of the grooves 1171 will be described as bottom surfaces 1173 (FIG. 8), and side surfaces of the grooves 1171 will be described as side surfaces 1174 (FIG. 8). Furthermore, in an embodiment, as described above, the second seal strengthening portions 117 can be configured by laser processing, and therefore, each of the top surfaces 1172, the bottom surfaces 1173, and the side surfaces 1174 is formed in an uneven shape.

Even when the configuration of the fourth modification as described above is adopted, it can achieve effects described above.

### Fifth Modification

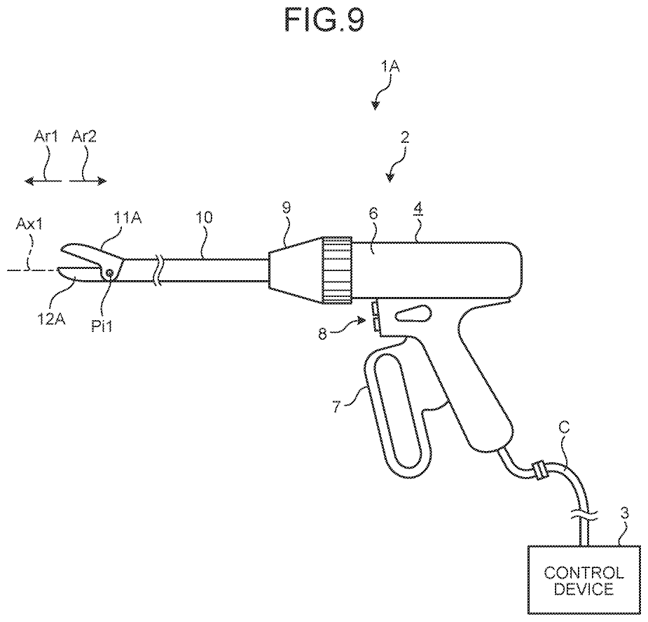

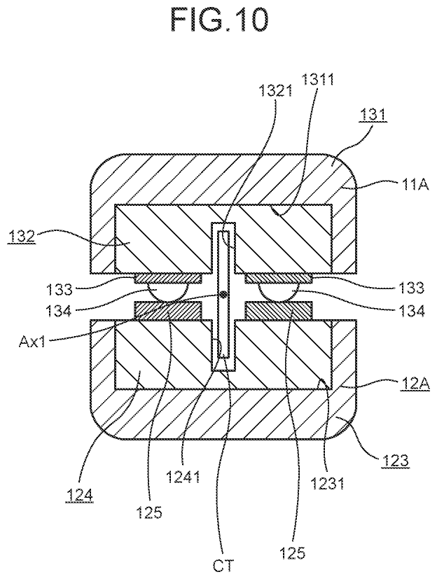

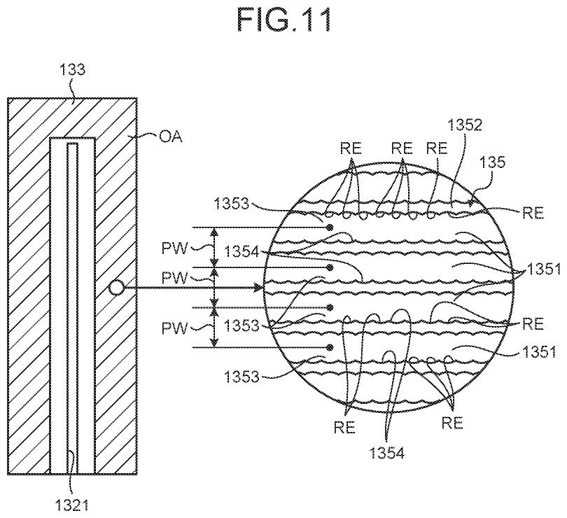

FIG. 9 to FIG. 11 are diagrams for explaining a fifth modification of an embodiment. Specifically, FIG. 9 is a diagram corresponding to FIG. 1, and illustrates a treatment system 1A according to the first modification. FIG. 10 is a diagram corresponding to FIG. 3 and is a cross-sectional view of a first jaw 11A and a second jaw 12A cut along a plane that is perpendicular to the central axis Ax1. FIG. 11 is a diagram of a first electrode 133 viewed at the side of the second jaw 12A.

In the treatment system 1A according to the fifth modification, as illustrated in FIG. 9 or FIG. 10, the first jaw 11A can be adopted instead of the jaw 11, and the second jaw 12A can be adopted instead of the vibration transmitter 12.

The first jaw 11A can include, as illustrated in FIG. 10, a first jaw main body 131, a first support member 132, the first electrode 133, and a contact portion 134.

The first jaw main body 131 can be formed in an elongated shape that extends along the central axis Aₓ₁. Further, an end portion of the first jaw main body 131 on the proximal end side Aᵣ₂ can be pivotally supported by the first pin Pᵢ₁ with respect to the sheath 10. Furthermore, the first jaw 11A can rotate about the central axis of the first pin Pᵢ₁ in conjunction with movement of the open-close mechanism D1 to the distal end side Aᵣ₁ or the proximal end side Aᵣ₂ in accordance with an open-close operation performed by the operating person on the operation handle 7 and can be opened and closed with respect to the second jaw 12A. A part of the first jaw main body 131 can be made of a metal material, such as stainless or titanium, to ensure predetermined rigidity.

In the first jaw main body 131, on a surface at the side of the second jaw 12A, as illustrated in FIG. 10, a recessed portion 1311 located at the center in the width direction and that extends along the central axis Aₓ₁ can be arranged.

The first support member 132 can be an elongated flat plate that extends along the central axis Aₓ₁ and can have an outer shape that is approximately the same as an inner shape of the recessed portion 1311. Further, the first support member 132 can be fitted to the recessed portion 1311. The first support member 132 can be configured with, for example, an electrically insulating material, such as PEEK, that has low thermal conductivity. Furthermore, the first support member 132 can be arranged between the first electrode 133 and the first jaw main body 131. In other words, by arranging the first support member 132, the first jaw main body 131 and the first electrode 133 are electrically insulated from each other.

In the first support member 132, in an approximately center portion in the width direction on a surface at the side of the second jaw 12A, as illustrated in FIG. 10, a cutter groove 1321 that extends along the central axis Ax1 can be arranged.

The first electrode 133 can be a portion that is configured with an electrically conductive material and high-frequency power can be supplied from a power supply to a portion between the first electrode 133 and a second electrode 125 (FIG. 10) that constitutes the second jaw 12A under the control of the control device 3. The first electrode 133 can be a flat plate with a U-shape that surrounds the cutter groove 1321 in flat manner. Further, both ends of the U-shape of the first electrode 133 can be fixed to the surface of the first support member 132 at the side of the second jaw 12A so as to be oriented toward the proximal end side Aᵣ₂.

Furthermore, a seal strengthening portion 135 can be arranged in a part of a surface of the first electrode 133 at the side of the second jaw 12A as illustrated in FIG. 11. In the fifth modification, the seal strengthening portion 135 can be arranged in a region 1331 that is located outside of the U-shape on the surface of the first electrode 133 at the side of the second jaw 12A. Meanwhile, the position at which the seal strengthening portion 135 is arranged can be located in a region other than the region 1331 as described above.

The seal strengthening portion 135 can be a portion that is formed in an uneven shape. In the fifth modification, the seal strengthening portion 135 can be formed by arranging a plurality of circular recessed portions RE in the region 1331 by laser processing using a short pulse laser as illustrated in FIG. 11. In the example illustrated in FIG. 11, a plurality of rows of the recessed portions RE (hereinafter, described as grooves 1351), in each of which the plurality of recessed portions RE can be arranged in an overlapping manner (in a communicating manner) in a direction perpendicular to the longitudinal direction of the first jaw 11A, can be arranged parallel to one another along the longitudinal direction.

Furthermore, in the fifth modification, each of depth dimensions and width dimensions of the grooves 1351 and interval dimensions (pitch widths PW (FIG. 11)) between the plurality of grooves 1351 can be set to be equal to or smaller than 100 μm. Meanwhile, the depth dimensions, the width dimensions, and the pitch widths PW of the grooves 1351 can be set to the same value or different values.

Meanwhile, in a region OA, portions other than the portions in which the grooves 1351 are formed will be described as a top surface 1352 (FIG. 11), bottom surfaces of the grooves 1351 will be described as bottom surfaces 1353 (FIG. 11), and side surfaces of the grooves 1351 will be described as side surfaces 1354 (FIG. 11).

Furthermore, in the fifth modification, as described above, the seal strengthening portion 135 can be configured by laser processing, and therefore, each of the top surface 1352, the bottom surfaces 1353, and the side surfaces 1354 can be formed in an uneven shape.

Moreover, in the first electrode 133, on the surface at the side of the second jaw 12A, a coating layer that is the same as the first coating layer CO₁ can be arranged.

The contact portion 134 is made of an electrically insulating material, can include a hemispherical shape, and can be arranged on the surface of the first electrode 133 at the side of the second jaw 12A. Further, the contact portion 134 can come into contact with or make contact with the second electrode 125 when the first jaw 11A is closed with respect to the second jaw 12A. In other words, the contact portion 134 can prevent a short circuit between the first electrode 133 and the second electrode 125.

The second jaw 12A can include, as illustrated in FIG. 10, a second jaw main body 123, a second support member 124, and the second electrode 125. The second jaw main body 123 is a portion in which a part of the sheath 10 can be extended toward the distal end side Aᵣ₁ and can be formed in an elongated shape that extends along the central axis Aₓ₁. In the second jaw main body 123, on a surface at the side of the first jaw 11A, as illustrated in FIG. 10, a recessed portion 1231 located at the center in the width direction and that extends along the central axis Aₓ₁ can be arranged.

The second support member 124 can be an elongated flat plate that extends along the central axis Aₓ₁ and can have an outer shape that is approximately the same as an inner shape of the recessed portion 1231. Further, the second support member 124 can be fitted to the recessed portion 1231. The second support member 124 can be configured with, for example, an electrically insulating material, such as PEEK, that has low thermal conductivity. Furthermore, the second support member 124 can be arranged between the second electrode 125 and the second jaw main body 123. In other words, by arranging the second support member 124, the second jaw main body 123 and the second electrode 125 can be electrically insulated from each other.

In the second support member 124, in an approximately center portion in the width direction on a surface at the side of the first jaw 11A, as illustrated in FIG. 10, a cutter groove 1241 that extends along the central axis Aₓ₁ and that faces the cutter groove 1321 when the first jaw 11A is closed with respect to the second jaw 12A can be arranged.

The second electrode 125 is a portion that can be configured with an electrically conductive material and high-frequency power can be supplied from a power supply to a portion between the second electrode 125 and the first electrode 133 under the control of the control device 3. The second electrode 125 can be a flat plate that has a U-shape that surrounds the cutter groove 1241 in flat manner. Further, both ends of the U-shape of the second electrode 125 can be fixed to the surface of the second support member 124 at the side of the first jaw 11A so as to be oriented toward the proximal end side Aᵣ₂.

Meanwhile, in the second electrode 125, on a surface at the side of the first jaw 11A, a coating layer that is the same as the first coating layer CO₁ can be arranged.

Furthermore, when applying high-frequency energy to the treatment target, the control device 3 can supply, via the electrical cable C, high-frequency power between the first electrode 133 and the second electrode 125 such as from a power supply. Accordingly, a high-frequency current can flow to the treatment target gripped between the first electrode 133 and the second electrode 125. In other words, the high-frequency energy can be applied to the target region.

Moreover, in the fifth modification, in the treatment tool 2, as illustrated in FIG. 10, a cutter CT located in the cutter grooves 1321 and 1241 and that moves back and forth along the central axis Aₓ₁ in accordance with operation of the treatment tool performed by the operating person, on an operating lever can be arranged. In other words, an incision can be made in the treatment target gripped between the first jaw 11A and the second jaw 12A by back-and-forth movement of the cutter CT.

Even when the configuration of the fifth modification as described above is adopted, it can achieve effects described above.

According to a treatment tool of the disclosure, it is possible to improve treatment performance.

Additional advantages and modifications will readily occur to those skilled in the art. Therefore, the disclosure in its broader aspects is not limited to the specific details and representative embodiments shown and described herein. Accordingly, various modifications can be made without departing from the spirit or scope of the general inventive concept as defined by the appended claims and their equivalents.

The above detailed description includes references to the accompanying drawings, which form a part of the detailed description. The drawings show, by way of illustration, specific embodiments that can be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

Example 1. A treatment tool comprising:
a vibration transmitter that has an elongated shape, the vibration transmitter being configured to treat a treatment target by ultrasonic vibration; and
a jaw configured to be opened and closed with respect to a treatment portion configured to treat the treatment target by the ultrasonic vibration of the vibration transmitter, the jaw being configured to grip the treatment target with the treatment portion, wherein
the treatment portion is configured to function as a first electrode between the first electrode and a second electrode configured to cause a high-frequency current to flow through the treatment target,
the jaw includes the second electrode, and
a first seal strengthening portion is arranged in at least a part of a surface of the treatment portion, the first seal strengthening portion being formed in an uneven shape to increase a contact surface with the treatment target to effectively supply the high-frequency current to the treatment target.

Example 2. The treatment tool according to Example 1, wherein the first seal strengthening portion includes a plurality of grooves each extending in a direction that crosses a longitudinal direction of the vibration transmitter.

Example 3. The treatment tool according to Example 1, wherein
the first seal strengthening portion includes a plurality of grooves each extending in a predetermined direction, and
each of the grooves is a groove in which a plurality of recessed portions formed by laser processing overlap with one another in the predetermined direction.

Example 4. The treatment tool according to Example 1, wherein in a surface of the vibration transmitter, a back surface opposite to a surface that faces the jaw is covered by an electrically insulating material.

Example 5. The treatment tool according to Example 4, wherein in the surface of the vibration transmitter, at least a part of a side surface adjacent to the back surface is covered by an electrically insulating material.

Example 6. The treatment tool according to Example 4, wherein the electrically insulating material is resin that include both of an ether group and a ketone group.

Example 7. The treatment tool according to Example 1, wherein at least a part of the surface of the treatment portion is covered by a water-repellent material.

Example 8. The treatment tool according to Example 7, wherein the water-repellent material is one of a fluorine coating agent and a siloxane coating agent.

Example 9. The treatment tool according to Example 1, wherein a depth dimension of the uneven shape of the first seal strengthening portion is equal to or smaller than 100 micrometers (μm).

Example 10. The treatment tool according to Example 1, wherein the first seal strengthening portion is arranged in a first non-contact portion that is adjacent to a first contact portion, on the surface of the treatment portion, the first non-contact portion being in no contact with the jaw when the jaw is closed with respect to the treatment portion, the first contact portion being in contact with the jaw when the jaw is closed with respect to the treatment portion.

Example 11. A treatment tool comprising:
a vibration transmitter that has an elongated shape, the vibration transmitter being configured to treat a treatment target by ultrasonic vibration; and
a jaw configured to be opened and closed with respect to a treatment portion configured to treat the treatment target by the ultrasonic vibration of the vibration transmitter, the jaw being configured to grip the treatment target with the treatment portion, wherein
the treatment portion is configured to function as a first electrode between the first electrode and a second electrode configured to cause a high-frequency current to flow through the treatment target,
the jaw includes the second electrode, and
a second seal strengthening portion is arranged in at least a part of a surface of the second electrode, the second seal strengthening portion being formed in an uneven shape to increase a contact surface with the treatment target to effectively supply the high-frequency current to the treatment target.

Example 12. The treatment tool according to Example 11, wherein the second seal strengthening portion is arranged in a second non-contact portion that is adjacent to a second contact portion, on a surface of the second electrode, the second non-contact portion being in no contact with the jaw when the jaw is closed with respect to the treatment portion, the second contact portion being in contact with the jaw when the jaw is closed with respect to the treatment portion.

Example 13. The treatment tool according to Example 1, further comprising:
a handle configured to perform an operation of opening and closing the jaw with respect to the treatment portion.

Example 14. A treatment tool comprising:
a first jaw that includes a first electrode between the first electrode and a second electrode configured to cause a high-frequency current to flow through the treatment target; and
a second jaw that includes the second electrode, the second jaw being configured to grip the treatment target with the first jaw, wherein
a seal strengthening portion that is formed in an uneven shape to increase a contact surface with the treatment target to effectively supply the high-frequency current to the treatment target is arranged in a part of surfaces of the first electrode and the second electrode.

What is claimed is:

1. A treatment tool comprising:
a first jaw including a first electrode; and
a second jaw configured to be opened and closed with respect to the first jaw, and the second jaw including a second electrode,
wherein the first electrode and the second electrode are configured to cause a high-frequency current,
wherein at least a part of the first jaw includes a first portion having an uneven surface.

2. The treatment tool according to claim 1, wherein the first portion includes a plurality of grooves each extending in a direction intersecting a longitudinal direction of the first jaw.

3. The treatment tool according to claim 2, wherein each groove of the plurality of grooves has side surfaces having a plurality of recesses recessed in the direction intersecting the longitudinal direction.

4. The treatment tool according to claim 1, wherein the first jaw comprises a first surface and a second surface,
the first surface configured to face the second jaw, and the first surface has the first portion, and
the second surface is configured not to face the second jaw, and the second surface has an electrically insulating material.

5. The treatment tool according to claim 4, wherein the first surface comprises a top surface, a first and a second inclined surfaces, the first and second inclined surfaces are inclined with respect to the top surface.

6. The treatment tool according to claim 4, wherein the electrically insulating material is resin that includes both of an ether group and a ketone group.

7. The treatment tool according to claim 4, wherein at least a part of the first surface has a water-repellent material.

8. The treatment tool according to claim 7, wherein the water-repellent material is at least one of a fluorine coating agent or a siloxane coating agent.

9. The treatment tool according to claim 1, wherein a thickness of the first portion is equal to or smaller than 100 micrometers (μm).

10. The treatment tool according to claim 4, wherein the first jaw comprises a third surface connecting the first surface and the second surface, the third surface does not contact with the second jaw when the second jaw is closed with respect to the first jaw, and wherein the first surface contacts with the second jaw when the second jaw is closed with respect to the first jaw.

11. The treatment tool according to claim 1, wherein the uneven surface comprises a first uneven surface, and
at least a part of the second jaw includes a second portion having a second uneven surface.

12. The treatment tool according to claim 11, wherein the second electrode comprises a first surface and a second surface,
the first surface configured not to contact with the first jaw when the second jaw is closed with respect to the first jaw,
the second surface configured not to contact with the first jaw, the second surface has the second portion when the second jaw is closed with respect to the first jaw.

13. The treatment tool according to claim 1, further comprising:
a handle configured to open and close the second jaw with respect to the first jaw.

14. The treatment tool according to claim 1, further comprising:
a vibration transmitter configured to treat a treatment target by ultrasonic vibration.

15. The treatment tool according to claim 2, wherein the plurality of grooves comprises a plurality of first grooves, the direction is a first direction,
the first jaw includes a second portion, the second portion includes a plurality of second grooves each extending in a second direction intersecting the longitudinal direction of the first jaw,
the first direction is symmetric with the second direction with respect to a longitudinal axis of the first jaw.

16. The treatment tool according to claim 2, wherein the direction is orthogonal to the longitudinal direction.

17. The treatment tool according to claim 5, wherein the first and second inclined surfaces have the first portion.

18. The treatment tool according to claim 5, wherein the top surface has the first portion.

19. The treatment tool according to claim 2, wherein the plurality of grooves comprises a top surface, a bottom surface, and a side surface connecting the top surface and the bottom surface, and
an area of the top surface is smaller than an area of the bottom surface.

20. The treatment tool according to claim 1, wherein the first jaw is configured to move with respect to the second jaw.
